(19) United States
(12) Patent Application Publication
(10) Pub. No.: US 2026/0157907 A1
(43) Pub. Date: Jun. 11, 2026

(54) CERVICAL SPINE POSITIONING APPARATUS AND METHOD

(71) Applicant: Marlin Dusty Richardson, Billings, MT (US)

(72) Inventor: Marlin Dusty Richardson, Billings, MT (US)

(21) Appl. No.: 19/179,764

(22) Filed: Apr. 15, 2025

Related U.S. Application Data
(60) Provisional application No. 63/633,961, filed on Apr. 15, 2024.

(51) Int. Cl. A61G 13/12; A61B 90/00

(52) U.S. Cl. CPC A61G 13/121; A61B 90/39; A61G 13/1295; A61B 2090/3966

(57) ABSTRACT

An apparatus and method are disclosed for positioning a medical patient's head relative to the patient's torso for use, for example, in preparing a patient for a spinal fusion surgery. The apparatus includes a support frame and connectors to removably attach the frame to the patient's head, including first and second lateral connectors that connect to sides of the patient's head, proximate the patient's ears, and a rear connector to connect to a rear portion of the patient's proximate the patient's inion. A leveling apparatus connected to the support frame indicates whether the patient's head is level relative to the patient's torso. The method includes attaching the apparatus to a patient in a prone position on a surgical table or elsewhere and positioning the patient's head in a neutral position based on information provided by the level apparatus.

### CROSS-REFERENCE TO RELATED APPLICATIONS

This application claims priority to U.S. Patent Application No. 63/633,961 entitled CERVICAL SPINE POSITIONING APPARATUS AND METHOD, pending, the disclosure of which is incorporated herein in its entirety by this reference.

### TECHNICAL FIELD

The present disclosure relates generally to a positioning apparatus for use by medical providers to position a patient and a method for using the same. More particularly, it relates to an apparatus for accurately positioning a patient's head relative to the patient's body when the patient is in a generally prone position, for example, during spine and neck surgery.

### BACKGROUND

In the field of health care and particularly in the field of neurosurgery, it is often necessary or desirable to position a patient in a prone position on a surgical table. In some surgical applications, cervical spine positioning is critical to ensure that the patient's head is set in a neutral position relative to the patient's body. For example, in the case of surgical vertebrae fusion, it is critical that the patient's head not be tilted (e.g., flexed, extended, rotated, or laterally bent) to the left or right or up or down, because a goal of surgery will limit the patient's range of head movement.

Various methods of cervical alignment fail to allow medical providers to quickly and accurately align the cervical spine. Some surgical tables allow adjustment of the patient's cervical alignment after the patient has been positioned prone on the table. Other techniques use one or more positioning cushions. One limitation of these techniques is that they depend upon the skill of the operator to set and maintain a good cervical position without objective feedback or references for precise positioning. Other techniques use clamps that physically attach to the patient's skull, which, while invasive, are commonly used and hold the patient's head securely.

### SUMMARY

The present invention is directed to an apparatus for positioning a patient's head relative to the patient's torso for use, for example, in preparing a patient for a spinal fusion surgery. The apparatus includes a support frame and connectors to removably attach the support frame to the patient's head. The connectors include first and second lateral connectors that are adapted to connect to sides of the patient's head, proximate the patient's ears and a rear connector adapted to connect to a rear portion of the patient's head proximate the patient's inion. A leveling apparatus connected to the support frame indicates whether the patient's head is flexed, extended, or laterally rotated relative to the ground. With the patient's torso being positioned horizontally, this allows for positioning of the patient's line of sight perpendicular to the ground. Particularly, the leveling apparatus indicates whether the patient's head is positioned in a neutral gaze, considering the rotational position and the flexion-extension position of the patient's head relative to a torso of the patient.

The present invention is also directed to a method of positioning a patient's head relative to the patient's torso, for example, in preparation for a spinal fusion surgery. The method includes securing a patient positioning apparatus to a head of the patient, wherein the positioning device includes a support frame and a level apparatus. The apparatus is secured to the patient's head at side connectors that contact the patient's head proximate the patient's ears and a rear connector that contacts a rear portion of the patient's head proximate the inion. The method further includes using the level apparatus to adjust the patient's head to a neutral gaze relative to the patient's torso, considering the rotational position and the flexion-extension position of the patient's head relative to a torso of the patient.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 shows a perspective view of one embodiment of the apparatus of the present invention.

FIG. 2 shows a top view of the embodiment of the apparatus of FIG. 1 in use connected to the head of a patient.

FIG. 3 shows a side view of the embodiment of the apparatus of FIGS. 1-2 positioned along the rear side of a patient's head.

FIG. 4 shows a perspective view of the components of an alternative embodiment of a connector.

FIG. 5 shows another perspective view of the components of the embodiment of the connector depicted in FIG. 4.

FIG. 6 shows a top view of the back end of the connector of FIGS. 4 and 5.

FIG. 7 shows an assembly profile view of the connector shown in FIGS. 4-6.

FIG. 8 shows a profile view of the assembled connector shown in FIGS. 4-7.

FIG. 9A-9I show examples of cushions that may be affixed to the connectors to contact the sides and rear of the patient's head.

### DETAILED DESCRIPTION

This description provides examples, and is not intended to limit the scope, applicability or configuration of the invention. Rather, the ensuing description will provide those skilled in the art with an enabling description for implementing embodiments of the invention. Various changes may be made in the function and arrangement of elements.

Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following systems, methods, devices, and software may individually or collectively be components of a larger system (e.g. optical or electromagnetic navigation systems), wherein other procedures may take precedence over or otherwise modify their application.

The present disclosure is directed to a patient positioning apparatus and method. In particular, the present disclosure is directed to an apparatus that aligns a patient's head relative to the patient's torso for use, for example, to align the patient's head prior to a cervical fusion surgery.

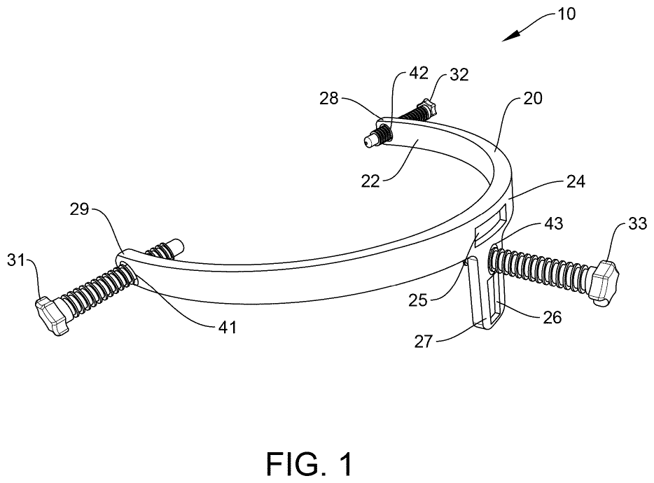

Referring now to FIG. 1, an example patient positioning apparatus 10 is disclosed. The apparatus 10 includes a support frame 20 adapted to wrap around a portion of a patient's head and be secured thereto. The support frame 20 in this example has an inner surface 22 and an outer surface 24 and has a generally semicircular shape. In use, the support frame 20 wraps around a portion of the patient's head with the inner surface 22 being proximate to the patient's head and the outer surface 24 generally facing away from the patient's head.

The apparatus 10 includes first and second side connectors 31, 32 proximate first and second ends 29, 28 of the support frame 20, respectively. The side connectors 31, 32 are designed to contact the patient's head (external auditory canal) and to removably secure the support frame 20 to the patient's head. In the example of FIG. 1, the side connectors 31, 32 are threaded screws connected, respectively, to first and second ends 29, 28, of the support frame 20 through first and second threaded apertures 41, 42. In use, the support frame 20 is positioned around a patient's head, the connectors 31, 32 are positioned at first and second sides of the patient's head, proximate the patient's ears. The side connectors 31, 32 are adjusted to contact the patient's ears such that the support frame is secured to the patient's head.

The apparatus 10 further includes a rear connector 33 that removably secures the support frame 20 to a rear portion of the patient's head proximate the patient's inion. The inion is the palpable bump on the midline of the occipital region. The rear connector 33 is positioned at a flange 27 of the support frame 20 that extends laterally from a center portion of the support frame 20. The rear connector 33 in the example of FIG. 1 is a threaded screw that connects to the support frame 20 through a rear threaded aperture 43. The length of the rear connector 33 and the location of the rear connector 33 relative to the flange 27 and the support frame 20 is based on anatomic measurements designed to keep the support frame 20 in plane and in line with the patient's line of sight. The rear connector 33 positions the patient's head in a neutral position (i.e., with visual gaze directed perpendicular to the floor when the patient is lying face down in a prone position on an operating table) when the rear connector 33 contacts the patient's head proximate the patient's inion. In one embodiment (not shown), the rear aperture 43 forms a slot that allows adjustment of the position of the rear connector 33 relative to the flange 27 to accommodate differences in patient anatomy.

The side connectors 31, 32 and the rear connector 33 may include a reusable or disposable cap (not shown) formed from a soft foam or other suitable material that allows the side connectors 31, 32 to securely fit into the patient's ears, referencing them to the aperture of the external auditory canal.

The support frame 20 of the apparatus 10 further includes a first recess 25 that is designed to hold a first level (not shown), which may be a common bubble level that indicates the relative lateral position of the patient's head when using the apparatus 10 (i.e., the patient's head positioned left or right relative to the torso). The support frame 20 further includes a second recess 26 that is designed to hold a second level (not shown) that is used to position the patient's head in a medial position relative to the patient's torso (i.e., up and down relative to the patient's torso).

In use, the apparatus 10 allows a surgeon to position a patient's head in a neutral position while the patient is secured in a skull clamp, such as a MAYFIELD head clamp, while prone on an operating table. By way of example, the apparatus 10 may be used in cases where the patient is undergoing fusion of the cervical spine from a posterior approach and is particularly useful in fusion of C1 and C2. Prior to a cervical fusion or other surgery, the patient is positioned on an operating table. The apparatus 10 is connected to the patient at first and second ears, using the side connectors 31, 32. The apparatus 10 is further connected to a rear portion of the patient's head proximate the patient's inion using the rear connector 33. The patient's head is then adjusted based on the first and second levels (not shown) positioned in the first and second recesses 25, 26 of the support frame, such that the patient's head is positioned generally straight forward relative to the patient's body, both laterally and medially.

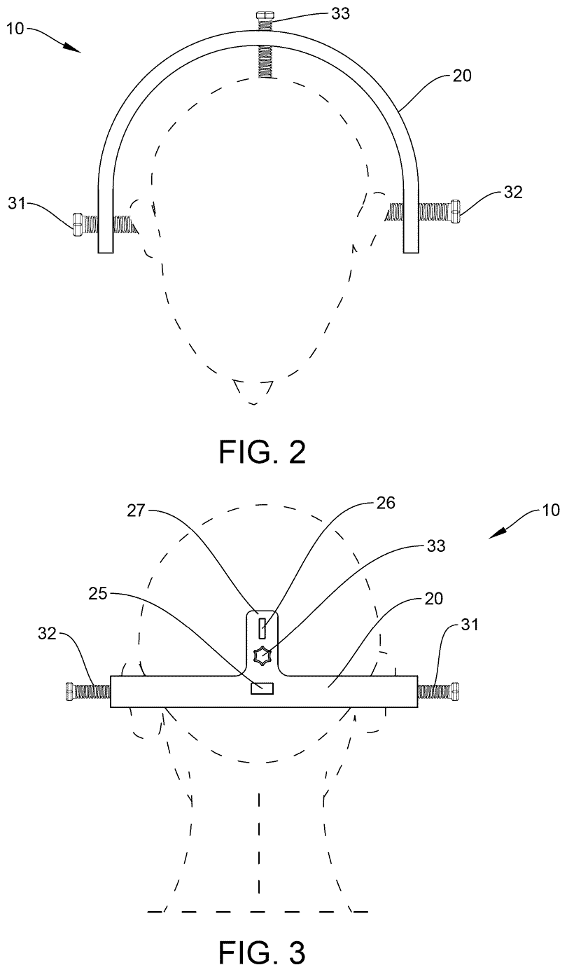

FIG. 2 shows a top view of the apparatus 10 in use connected to the head of a patient. Side connectors 31, 32 attach removably from the support frame 20 to opposing sides of the patient's head, proximate the patient's ears. In one embodiment, the support frame 20 is generally flexible and tensioned such that it urges the first and second side connectors 31, 32 toward the patient's head. The apparatus 10 further includes a rear connector 33 that adjustably connects to a rear portion of the patient's head proximate the patient's inion. In use, the apparatus 10 is positioned generally around the patient's head. The connectors 31, 32, 33 are adjusted to contact the patient's head and to removably secure the apparatus 10 to the patient's head.

FIG. 3 shows a top view of the apparatus 10 positioned along the rear side of a patient's head. First and second connectors 31, 32 adjustably connect to the patient's head, proximate the patient's ears. The rear connector 33 adjustably connects to a rear portion of the patient's head, proximate the patient's inion. Recesses 25, 26 in the support frame 20 are adapted to hold levels (not shown) such as common liquid bubble levels to allow the head of the patient to be positioned in a neutral position relative to the patient's torso. When the apparatus 10 is properly fitted to the patient, the leveling bubbles will focus the patient's line of sight perpendicular to the floor in a “neutral gaze.” That is to say, the patient's head would not be tilted and would neither be looking up or down relative to the patient's torso.

In an alternative embodiment, the leveling system uses more technologically advanced measurement systems rather than a simple bubble level. For example, the leveling system can be configured to work with other alignment techniques using stereotactic infrared or electromagnetic navigation or x-ray fiducial markers. Using visual, infrared, or other stereotactic fiducial points, the apparatus can be referenced to other anatomic structures and use stereotactic navigation to give live updates and greater accuracy

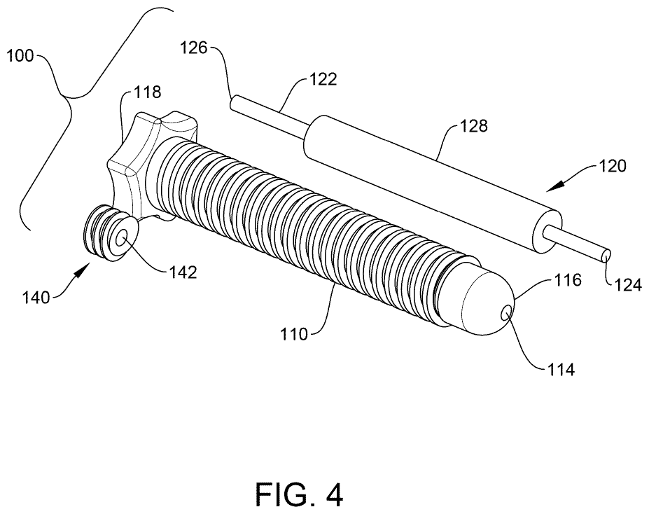

FIG. 4 shows a perspective view of the components of an alternative embodiment of a connector 100, that can be used as a side connector (31, 32 in FIG. 1) or rear connector (33 in FIG. 1.). The connector 100 in this embodiment includes a screw 110 that defines a hollow chamber (not shown in FIG. 4) that functions as a piston wall. The connector 100 further includes a cylindrical sliding pin assembly 120 that includes a pin 122 having first and second ends 124, 126 that is generally centered along a central axis of the sliding pin assembly 120, connected to a spring mechanism 128 that urges the pin 120 to move longitudinally along the central axis. The pin assembly 120 is disposed and secured within the chamber of the screw 110. The first end 124 of the pin 122 extends from the hollow chamber (not shown) of the screw 110 through an aperture 114 in the screw 110. A cap 140 secures the pin assembly 120 within the chamber and contacts the second end 126 of the pin 122. The cap 140 includes an aperture 142 through which the second end 126 of the pin 122 protrudes.

In use, the pin assembly 120 indicates when the connector 100 is tight on the patient. In a resting position, the first end 124 of the pin 122 protrudes through the aperture 114 in screw 110. As the screw 110 is tightened relative to the support frame 20, the first end 124 of the pin 122 is urged toward the front end 116 of the screw 110 until the first end 124 is flush with the front end 116. A cushion (not shown) is disposed at the front end 116. As the screw 110 seats, the second end 126 of the pin 122 protrudes from the back end 118 of the screw 110 through the aperture 142 in the cap 140. This protrusion provides a user (e.g., a surgeon) a visual cue as to the tightness of the connector 100 on the patient.

In one embodiment, the connectors 100 include visual indicators to indicate their tightness on the patient. For example, the outside of the threaded screw 110 may be colored in two or more different colors to indicate to the user the depth of the screw 110 relative to the frame 20. In another example, screw 110 may include stops (not shown) or other raised portions (not shown) to visually indicate the depth of the screw 110 relative to the frame 20.

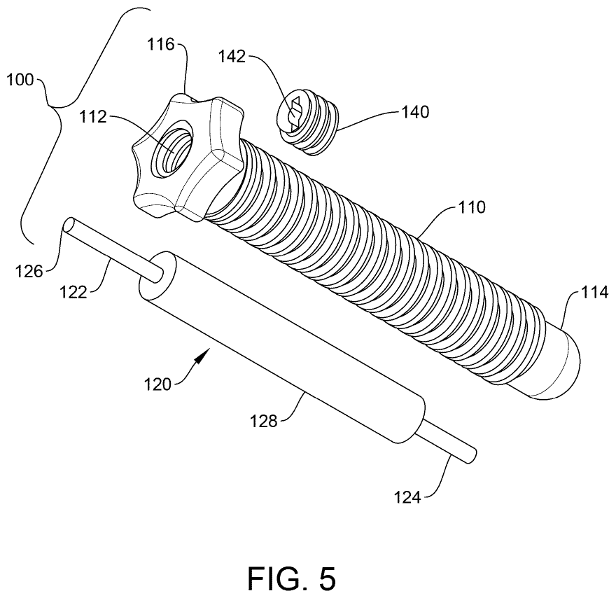

FIG. 5 shows another assembly view of the components of the embodiment of the connector 100 depicted in FIG. 4. The hollow chamber 112 is defined within the screw 110 and is generally cylindrical, running along a central axis of the screw 110. The pin assembly 120 fit within the chamber 112 of the screw 110. As shown, the cap 140 includes an aperture 142. The cap 140 secures the pin assembly 120 within the hollow chamber 112 of the screw 110. The second end 126 of the pin 122 is positioned within the aperture 142 in the cap 140 and can extend through the aperture 142, depending upon the tension of the connector 100, particularly depending upon the tightness of the screw 110.

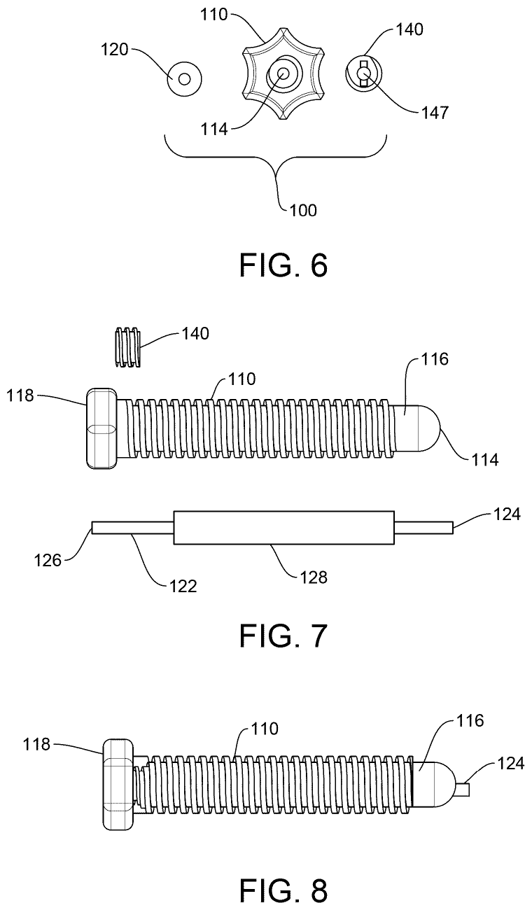

FIG. 6 shows a top view of the back end of the connector 100 of FIGS. 4-5.

FIG. 7 shows an assembly profile view of the connector 100 shown in FIGS. 4-6.

FIG. 8 shows a profile view of the assembled connector 100 shown in FIGS. 4-7. The pin assembly (120 in FIGS. 4-7) is disposed within the chamber (112 in FIG. 5) of the screw 110. The first end 124 of the pin 122 protrudes through the aperture (114 in FIGS. 4, 6) of the front end 116 of the screw 110 in this illustration.

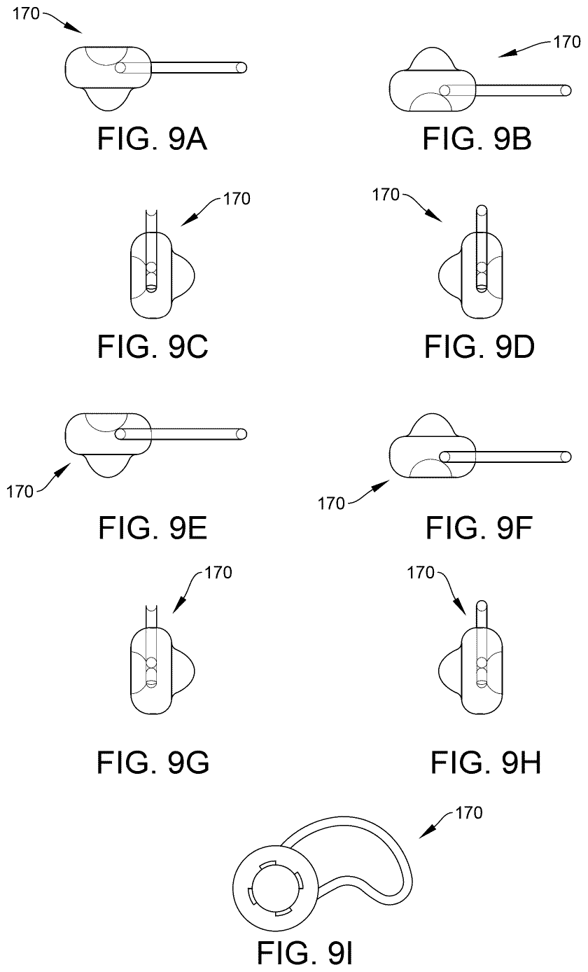

FIGS. 9A-9I show examples of cushions 170 that may be connected to connector to contact the sides and rear of the patient's head.

The description herein provides examples, and is not limiting of the scope, applicability, or examples set forth in the claims. Changes may be made in the function and arrangement of elements discussed without departing from the scope of the disclosure. Various examples may omit, substitute, or add various procedures or components as appropriate. Also, features described with respect to some examples may be combined in other examples.

The description set forth herein, in connection with the appended drawings, describes example configurations and does not represent all the examples that may be implemented or that are within the scope of the claims. The term “exemplary” as may be used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (e.g., A and B and C).

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

What is claimed is:

1. A patient positioning apparatus comprising:
a support frame;
first and second lateral connectors connected to the support frame, wherein said first and second connectors are positioned to adjustably contact first and second sides, respectively, of a patient's head proximate first and second ears on the patient's head;
a rear contact connected to the support frame, wherein the rear contact is positioned to adjustably contact a rear portion of the patient's head; and
a level apparatus attached to the support frame.

2. The apparatus of claim 1, wherein the support frame is flexible and urges first and second ends of the support frame proximate the first and second connectors, respectively, toward the first and second sides of the patient's head or body.

3. The apparatus of claim 1, wherein the level system includes a first level that relates to rotational position of the patient's head relative to a torso of the patient, and a second level that relates to flexion-extension position of the patient's head relative to the torso.

4. The apparatus of claim 1, wherein the level apparatus comprises first and second bubble levels.

5. The apparatus of claim 1, wherein the level apparatus comprises fiducial markers referenced to one or more anatomic structures on the patient's head.

6. The apparatus of claim 5, wherein the fiducial markers allow for use of visual or electromagnetic navigation.

7. The apparatus of claim 5, wherein the fiducial markers include x-ray markers.

8. The apparatus of claim 1, wherein the first and second connectors are screws that adjustably connect through first and second ends of the first and second sides, respectively, of the support frame.

9. The apparatus of claim 1, further comprising a wherein the support frame further comprises a flange located proximate a center portion of the support frame, that extends laterally from the support frame, and wherein the rear contact is connected to the support frame at the flange.

10. The apparatus of claim 9, wherein the rear contact comprises a screw connector that is adjustably connected to the flange of the support frame.

11. The apparatus of claim 9, wherein the rear connector adjustably connects to the flange through a slot that allows lateral adjustment of the rear connector relative to the flange.

12. A method of positioning a patient comprising:
attaching a patient positioning device to a head of the patient, wherein the positioning device includes
a support frame;
first and second lateral contacts connected to the support frame;
a rear contact connected to the support frame; and
a level apparatus attached to the support frame;
securing the first and second lateral contacts to first and second sides of patient's head, respectively, near first and second ears;
securing the rear contact to a rear portion of the patient's head; and
moving the patient's head to a desired position based upon information provided by the level apparatus.

13. The method of claim 12, where in the support frame is flexible and urges first and second ends of the support frame proximate the first and second connectors, respectively, toward the first and second sides of the patient's head or body.

14. The method of claim 12,
wherein the first and second lateral contacts adjustably connect to the support frame, and
wherein the securing the first and second lateral contacts comprises adjusting the first and second lateral contacts relative to the support frame.

15. The method of claim 14,
wherein the rear contact adjustably connects to the support frame, and
wherein the securing the rear contact comprises adjusting the rear contact relative to the support frame.

16. The method of claim 12, wherein the support frame further comprises a flange extending laterally from a center portion of the support frame, and wherein the rear contact connects to the flange.

17. The method of claim 16,
wherein the rear contact adjustably connects to a slot in the flange, and
wherein the securing the rear contact comprises positioning the rear contact relative to the slot and fixedly connecting the rear contact to the support frame after the positioning.

18. The method of claim 12,
wherein the first and second connectors adjustably connect to first and second ends, respectively, of the support frame, using threads having visual markers indicating adjustment of the first and second ends relative to the support frame, and
wherein the securing the first and second connectors comprises threading the first and second connectors through the first and second ends of the support frame based upon the visual markers.
